(19) United States
(12) Patent Application Publication
(10) Pub. No.: US 2015/0246065 A1
(43) Pub. Date: Sep. 3, 2015

(54) Topical Pharmaceutical Composition

(71) Applicant: CIPLA LIMITED, Mumbai (IN)

(72) Inventors: Geena Malhotra, Mumabi (IN); Shrinivas Madhukar Purandare, Mumbai (IN)

(21) Appl. No.: 14/427,906

(22) PCT Filed: Nov. 14, 2013
(86) PCT No.: PCT/GB2013/053001

§ 371 Date: Mar. 12, 2015

(30) Foreign Application Priority Data
Sep. 14, 2012 (IN) 2687/MUM/2012

(51) Int. Cl. A61K 31/688; A61K 45/06; A61K 31/4418; A61K 31/19; A61K 9/00; A61K 31/675

(52) U.S. Cl. CPC A61K 31/688; A61K 9/0014; A61K 31/675; A61K 31/4418; A61K 31/19; A61K 45/06; A61K 9/0034

(57) ABSTRACT

The present invention relates in general to a topical pharmaceutical composition comprising an antiretroviral agent in combination with a bactericidal agent and optionally an antifungal agent, particularly for use as a contraceptive.

### CROSS REFERENCE TO RELATED APPLICATIONS

This application is a filing under 35 U.S.C. 371 of International Application No. PCT/GB2013/053001 filed Nov. 14, 2013, entitled “Topical Pharmaceutical Composition,” which claims priority to Indian Patent Application No. 2687/MUM/2012 filed Sep. 14, 2012, which applications are incorporated by reference herein in their entirety.

### FIELD OF INVENTION

The present invention relates to a topical pharmaceutical composition comprising an antiretroviral agent in combination with a bactericidal agent and optionally an antifungal agent, particularly for use as a contraceptive. This invention also discloses a process of preparation of the said topical pharmaceutical composition, and certain uses of the composition.

### BACKGROUND AND PRIOR ART

Oral contraceptives are the most popular form of reversible contraception. Approximately 30% of women of reproductive age currently use oral contraceptives and 80% of all women will use oral contraceptives at some time during their reproductive years. Oral contraceptives provide both a high degree of contraceptive efficacy and a range of non-contraceptive health benefits such as decreased menstrual cramps, protection against ovarian and endometrial cancers, ectopic pregnancy and pelvic inflammatory disease. An estimated 1614 per 100,000 pill users currently avoid hospitalization because of the protective effects of oral contraceptives. The method also has an excellent safety profile that reflects a steady decline in the constituent components; estrogen and progestin.

However, oral contraceptives require the use of a barrier method for protection against sexually transmitted diseases which increase the prevalence of vaginitis caused by Candida species, require prolonged use regardless of the frequency of sexual intercourse, are relatively expensive, decrease libido, cause irreversible chloasma (patchy facial pigmentation) when users are exposed to the sun and also result in minor abnormalities such as elevated thyroxine levels.

Apart from oral contraceptives various other modes of contraception can be adopted ranging from physical methods to chemical methods and even surgical methods. Male and female condoms, diaphragms, intrauterine devices, vasectomy, tubectomy are few such methods.

Further, Acquired Immuno-Deficiency Virus (AIDS) is one of the most threatening and fatal diseases and has been now considered as a pandemic. It is caused by the Human Immunodeficiency Virus (HIV) which can be transferred or contracted in various ways but the primary modes are via unprotected sex and blood transfusions.

Contraception is a method which is used to prevent pregnancy but it equally serves the purpose preventing sexually transmitted diseases (STD), mainly AIDS, by preventing its transmission.

Vaginal infections (vaginitis) is commonly observed and is caused by Candida (yeast infection), Chlamydia (sexually transmitted disease, Gardnerella bacteria or gonorrhea. Most vaginal infections can be classified into: yeast infection, trichomoniasis, or bacterial vaginosis. Symptoms of these infections may include redness, swelling, irritation, itching and an unusual discharge or odour.

Such vaginal infections can be commonly treated with the use of antifungal tablets or creams.

However, none of the above mentioned methods have sufficient surety of preventing HIV transmission and there is always a minute chance of contracting AIDS. Certain drug moieties such as antiretrovirals or antifungal agents (Drug-Induced Reactivation of Apoptosis Abrogates HIV-1 Infection, Hartmut M. Hanauske-Abel et al, PLOS ONE, September 2013, Volume 8, Issue 9, e74414 have the ability to limit the HIV production by blocking its replication process. This in turn causes the virus concentration to steadily decrease.

Tenofovir is one such drug moiety and is used in its prodrug form of tenofovir disoproxyl fumarate (or PMPA) as an antiretroviral agent which is a nucleotide analogue reverse transcriptase inhibitor. Tenofovir is known to block reverse transcriptase which is essential in viral production thus preventing replication of viral cells resulting in the containment of the virus. It's chemically known as ({[(2R)-1-(6-amino-9H-purin-9-yl)propan-2-yl]oxy}methyl)phosphonic acid and its chemical structure is as follows.

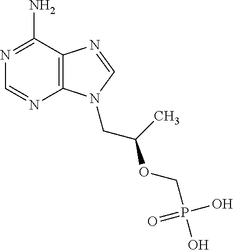 [SMILES: C[C@H](Cn1cnc2c(N)ncnc21)OCP(=O)(O)O]

Tenofovir is commercially available as Viread® which is in the form of tablets and oral powders. Truvada® is a combination of tenofovir with emtricitabine as a tablet and Atripla® is a combination of tenofovir with emtricitabine and efavirenz as a tablet. The recommended daily dose of tenofovir in these formulations ranges from 150 mg to 300 mg depending on the severity of the condition.

Ciclopirox is a broad-spectrum, antifungal agent and is chemically known as 6-cyclohexyl-1-hydroxy-4-methyl-2(1H)-pyridone, 2-aminoethanol salt.

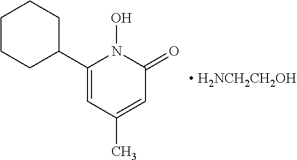

Ciclopirox is a hydroxypyridone antifungal agent that acts by chelation of polyvalent cations (Fe3+ or Al3+), resulting in the inhibition of the metal-dependent enzymes that are responsible for the degradation of peroxides within the fungal cell.

EP1773296 discloses a vaginal gel formulation comprising tenofovir which is used as an antiretroviral agent.

“In vaginal fluid, bacteria associated with bacterial vaginosis can be suppressed with lactic acid but not hydrogen peroxide” by Dierdre E O'Hanlon, Thomas R Moench and Richard A Cone, BMC Infectious Diseases 2011, 11: 200 discloses the use of lactic acid against bacterial vaginosis.

“Gels as vaginal drug delivery systems Review Article” International Journal of Pharmaceutics, Volume 318, Issues 1-2, 2 Aug. 2006, Pages 1-14 J. das Neves, M. F. Bahia.

US20050037033 discloses a microbicidal compositions containing ciclopirox olamine for preventing the transmission of or treating sexually transmitted infections and/or common vaginal infections.

WO9602226 discloses a pharmaceutical composition comprising a combination of 1-hydroxy-2-pyridones such as ciclopirox or octopirox and crotamiton as an antifungal agent activity enhancer.

WO9717075 discloses a topical foamable pharmaceutical composition of ciclopirox or ciclopirox olamine and surfactant for treating skin diseases induced by oval pityrosporum.

Reformulated tenofovir gel for use as a dual compartment microbicide, Charlene S. Dezzutti et al, J Antimicrob Chemotherapy, 1-4, 2012.

In Vitro and Ex Vivo Testing of Tenofovir Shows It Is Effective As an HIV-1 Microbicide, Lisa C. Rohan et al, PLoS ONE, February 2010, Volume 5, Issue 2, e9310.

The prior art discloses tenofovir for antiretroviral use in a vaginal gel formulation. Lactic acid for the treatment of bacterial vaginosis has also been disclosed in another prior art. There is prior art which also discloses formulations of ciclopirox for the treatment of fungal infections.

However, it is noted that none of the prior arts disclose tenofovir, lactic acid and ciclopirox in a combination. Moreover the use of this combination specifically as contraceptive agent has also not been disclosed through any of the prior arts.

We have appreciated there is a need for improved compositions for treating viral and bacterial infections of the aforementioned type. We have also appreciated the need for improved methods of facilitating or providing contraception.

### OBJECT OF THE INVENTION

The object of the present invention is to provide a topical pharmaceutical composition comprising tenofovir and at least one or more antibacterial agents and optionally ciclopirox.

Another object of the present invention is to provide a topical pharmaceutical composition comprising a combination of tenofovir and at least one or more antibacterial agent and optionally ciclopirox optionally with one or more pharmaceutic ally acceptable excipients.

Yet another object of the present invention is to provide a topical pharmaceutical composition comprising tenofovir and at least one or more antibacterial agents and optionally ciclopirox optionally with one or more pharmaceutically acceptable excipients for vaginal application.

Another object of the present invention is to provide a topical pharmaceutical composition comprising tenofovir and at least one or more antibacterial agents and optionally ciclopirox which ensures high efficacy.

Another object of the present invention is to provide a stable topical pharmaceutical composition comprising tenofovir and at least one or more antibacterial agents and optionally ciclopirox.

Another object of the present invention is to provide a process for preparing the topical pharmaceutical composition comprising tenofovir and at least one or more antibacterial agents and optionally ciclopirox.

Another object of the present invention is to provide a method of contraception by applying the topical pharmaceutical composition comprising tenofovir and at least one or more antibacterial agents and optionally ciclopirox.

Another object of the present invention is to provide a method of facilitating contraception, preventing the transmission of retroviral infections and the treatment of bacterial vaginosis by applying the topical pharmaceutical composition comprising tenofovir and at least one or more antibacterial agents and optionally ciclopirox.

Another object of the present invention is to provide use of the topical pharmaceutical composition comprising tenofovir and at least one or more antibacterial agents and optionally ciclopirox as a contraceptive agent.

Another object of the present invention is to provide use of the topical pharmaceutical composition comprising tenofovir and at least one or more antibacterial agents and optionally ciclopirox for facilitating contraception, preventing the transmission of retroviral infections and the treatment of bacterial vaginosis.

### SUMMARY OF THE INVENTION

According to one aspect of the present invention, there is provided a topical pharmaceutical composition comprising tenofovir and at least one antibacterial agent and, optionally, ciclopirox.

Preferably, the composition also comprises ciclopirox.

Tenofovir, the at least one antibacterial agent and ciclopirox may be in the form of a pharmaceutically acceptable derivative comprising, for example, pharmaceutic ally acceptable salts, solvates, complexes, hydrates, anhydrates, isomers, esters, tautomers, enantiomers, polymorphs, or prodrugs.

Suitably, the composition of the invention comprises one or more pharmaceutically acceptable excipients. The composition is preferably in the form of a gel. Vaginal gels—that is, those gels suitable for topical vaginal application—are particularly preferred.

The invention also provides, in another aspect, a process for preparing a topical pharmaceutical composition according to the invention in the form of a vaginal gel, which process comprises
a) preparing a drug phase comprising tenofovir and at least one antibacterial agent and along with one or more pharmaceutically acceptable excipients;
b) preparing a ciclopirox solution along with one or more pharmaceutically acceptable excipients;
c) dispersing ciclopirox solution of step b) into the drug phase of step a) to form the gel.

In a further aspect, the invention provides a method of providing or facilitating contraception which method comprises the application of a topical pharmaceutical composition according to the invention to a patient in need thereof.

The invention also provides a method of treating retroviral infections or bacterial infections, particularly bacterial vaginosis, which method comprises the application of a topical pharmaceutical composition according to the invention to a patient in need thereof.

In a further aspect of the invention, there is provided a topical pharmaceutical composition as defined herein for use as a medicament, in particular for use as a contraceptive agent, or to facilitate contraception.

The invention also provides a topical pharmaceutical composition as defined herein for use in treating retroviral infections, or bacterial infections, particularly bacterial vaginosis.

According to an aspect of the present invention, there is provided a topical pharmaceutical composition comprising tenofovir or any of its pharmaceutically acceptable derivatives and at least one or more antibacterial agents or any of its pharmaceutically acceptable derivatives and optionally ciclopirox or any of its pharmaceutically acceptable derivatives.

According to another aspect of the invention, there is provided a process for the preparation of a topical pharmaceutical composition comprising tenofovir and at least one or more antibacterial agents and optionally ciclopirox optionally with one or more pharmaceutically acceptable excipients.

According to yet another aspect of the present invention there is provided a method of contraception by applying a topical pharmaceutical composition comprising tenofovir and at least one or more antibacterial agents and optionally ciclopirox.

According to yet another aspect of the present invention there is provided a method of preventing the transmission of retroviral infections and the treatment of bacterial vaginosis by applying the topical pharmaceutical composition comprising tenofovir and at least one or more antibacterial agents and optionally ciclopirox.

According to yet another aspect of the invention there is provided use of the topical pharmaceutical composition comprising tenofovir and at least one or more antibacterial agents and optionally ciclopirox as a contraceptive agent.

According to yet another aspect of the invention there is provided use of the topical pharmaceutical composition comprising tenofovir and at least one or more antibacterial agents and optionally ciclopirox for preventing the transmission of retroviral infections and the treatment of bacterial vaginosis.

### DETAILED DESCRIPTION OF THE INVENTION

Contraception is used to prevent pregnancy as well as sexually transmitted diseases. A number of methods are available for contraception; each having their own advantages and disadvantages. Male and female condoms, female diaphragms, hormonal pills, intrauterine devices, vasectomy, tubectomy are some of the contraceptive methods but none of them provide sufficient guarantee or surety of being totally able to prevent pregnancy as well as contracting sexually transmitted diseases.

The inventors of the present invention have found that a combination of tenofovir and at least one or more antibacterial agents and optionally ciclopirox exhibits spermicidal tendencies due to acid-buffering properties when mixed with semen. Further, this combination amplifies the contraceptive effect thus negating the chances of pregnancy or transfer of such sexually transmitted diseases as well as the treatment of bacterial vaginosis.

According to the present invention, antibacterial agents, may include but are not limited to one or more of lactic acid, citric acid, fumaric acid, tartaric acid, malic acid, acetic acid, mixtures thereof and the like. Preferably, the antibacterial agent may be lactic acid. Two or more antibacterial agents may be used if desired.

The terms “Tenofovir”, “antibacterial agent” and “Cyclopirox”, are used in a broad sense to include not only “Tenofovir free base” or “Cyclopirox free base” etc per se but also their pharmaceutically acceptable derivatives. Suitable pharmaceutically acceptable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof.

The term “Lactic acid” is also used in broad sense to include not only “Lactic acid moiety” per se but also its pharmaceutically acceptable derivatives. Suitable pharmaceutically acceptable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof.

The composition of the invention may, for example, comprise lactic acid in the form of racemic lactic acid, D(−) lactic acid, or L(+) lactic acid, or a pharmaceutically acceptable salt of one of the free acid forms, or a hydrate or solvate of one of the free acid or salt forms. The same principle applies where applicable to other acids such as, for example, citric acid, fumaric acid, tartaric acid, malic acid, and acetic acid.

Preferably, lactic acid may be present in an amount ranging from about 1% to about 10% by weight of the total composition.

The term “Cyclopirox” is also used in broad sense to include not only “Cyclopirox” per se but also its pharmaceutically acceptable derivatives. Suitable pharmaceutically acceptable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof.

The term “pharmaceutically acceptable derivative” means a pharmaceutically active compound with equivalent or near equivalent physiological functionality when compared to the active moiety. As used herein, the term “pharmaceutically acceptable derivative” includes any pharmaceutically acceptable ether, stereoisomer including enantiomer, diastereomer or stereoisomerically enriched or racemic mixture and any other compound which upon administration to the recipient is capable of providing (directly or indirectly) such a compound or an antivirally active metabolite or residue thereof.

Examples of pharmaceutically acceptable salts of tenofovir and its pharmaceutically acceptable derivatives include salts derived from an appropriate base such as an alkali metal (for example, sodium), an alkaline earth metal (for example magnesium), ammonium and NX₄+ (wherein X is C₁-C₄ alkyl). Pharmaceutically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids such as hydrochloric, sulphuric, phosphoric and sulfamic acids. Pharmaceutically acceptable salts of a compound of a hydroxy group include the anion of said compound in combination with a suitable cation such as Na+ and NX₄+ (wherein X is independently selected from H or a C₁-C₄ alkyl group).

For therapeutic use, salts of active ingredients will be pharmaceutically acceptable i.e. they will be salts derived from a pharmaceutically acceptable acid or base. However, salts of acids or bases which are not physiologically acceptable may also find use, for example, in the preparation or purification of a pharmaceutically acceptable compound. All salts, whether or not derived from a pharmaceutically acceptable acid or base, are within the scope of the present invention.

The term “prodrug” as used herein refers to any compound that when administered to a biological system generates the drug substance i.e. active ingredient, as a result of spontaneous chemical reaction(s), enzyme catalyzed chemical reaction(s), and/or metabolic chemical reaction(s).

“Prodrug moiety” means a labile functional group which separates from the active inhibitory compound during metabolism systemically inside a cell by hydrolysis, enzymatic cleavage or by some other method. Prodrug moieties can serve to enhance solubility, absorption and lipophilicity which in turn improve drug delivery, bioavailability and efficacy. A “prodrug” is thus a covalently modified analog of a therapeutically active compound.

Tenofovir is highly efficacious when administered in any of its prodrug forms namely PMEA [9-(2-Phosphonylmethoxyethyl)-adenine)] or PMPA [(1R)-9-(2-Phosphonylmethoxypropyl)-adenine] or tenofovir disoproxyl fumarate or tenofovir alafenamide fumarate.

In one preferred aspect, tenofovir is in the form of tenofovir disoproxyl fumarate or tenofovir alafenamide fumarate.

Preferably, tenofovir may be present in an amount ranging from about 1% to about 5% by weight of the total composition.

Cyclopirox is preferably provided in the form of ethanolamine salt of ciclopirox, referred to as ciclopirox olamine. Thus, a preferred topical pharmaceutical composition according the invention comprises ciclopirox in the form of ciclopirox olamine.

Preferably, ciclopirox may be present in an amount ranging from about 0.05% to about 5% by weight of the total composition.

A particularly preferred topical pharmaceutical composition according to the invention preceding comprises tenofovir disoproxil fumarate and lactic acid and optionally ciclopirox olamine along with one or more pharmaceutically acceptable excipients.

Another particularly preferred topical pharmaceutical composition according to the invention comprises tenofovir alafenamide fumarate and lactic acid and optionally ciclopirox olamine along with one or more pharmaceutically acceptable excipients.

Preferably, in each case above, ciclopirox olamine is present in the topical pharmaceutical composition.

In a preferred aspect, the topical pharmaceutical composition of the present invention may comprise tenofovir disoproxil fumarate and lactic acid and optionally ciclopirox olamine in a dosage form suitable for vaginal application.

In another aspect, the topical pharmaceutical composition of the present invention may comprise tenofovir alafenamide fumarate and lactic acid and optionally ciclopirox olamine in the dosage form suitable for vaginal application.

The topical pharmaceutical composition of the present invention comprising tenofovir and lactic acid and optionally ciclopirox may be in the dosage form suitable for vaginal application such as, but not limited to gels, tablets, capsules, pessaries, tampons, creams, pastes, jellies, tablets, foams, films, rings, implants or sprays and the like. Preferably, the topical pharmaceutical composition according to the present invention may be in the form of a vaginal gel.

The topical pharmaceutical composition of the present invention comprising tenofovir and lactic acid and optionally ciclopirox may be in the form of controlled release formulation, delayed release formulation, extended release formulation, pulsatile release formulation, and mixed immediate release and controlled release formulation and the like. The composition is formulated such that it releases the active ingredient/s at a rate which will result in an effective concentration at the site of application.

General desirable aspects of topical pharmaceutical composition, preferably in the form of a vaginal gel include, safety (e.g. isosmolar aqueous gels), efficacy, stability, and patient acceptability. More specific formulation aspects include optimal retention time, appropriate drug diffusion, and targeted drug delivery.

A topical pharmaceutical composition according to the invention is preferably in a dosage form suitable for vaginal or rectal application. Thus, for example, the topical pharmaceutical composition may be provided in the form of a vaginal gel or rectal gel.

A topical pharmaceutical composition according to the invention may, however, be provided in the form of, for example, a gel, tablet, capsule, pessary, tampon, cream, paste, jelly, foam, film, ring, spray, suppository, enema, ointment, solution or suspension, as desired by the skilled person.

It will be understood that the pH of the composition can be varied by the skilled person as required. A topical pharmaceutical composition according to the invention preferably has a pH of from 3.0 to 4.5.

The topical pharmaceutical composition of the invention may, if desired, further comprise at least one antiretroviral agent selected from protease inhibitors, nucleoside reverse transcriptase inhibitors, nucleotide reverse transcriptase inhibitors, non-nucleotide reverse transcriptase inhibitors and integrase inhibitors. The skilled person will be aware of suitable agents which can be used.

The topical pharmaceutical composition according to the present invention in the form of a vaginal gel is easy to use, discreet, painless to the patient, cost effective and safe for continuous administration. The topical pharmaceutical composition according to the present invention in the form of a vaginal gel further allows self-administration, with minimal interference with body functioning and daily life.

Rheological properties of gels have considerable influence in the contraceptive success. As the consistency of the applied product increases, its efficacy may also increase as a result of becoming more tenacious and more resistant to sperm migration and consequently decreasing the capability of sperm to reach the site of fertilization.

According to an aspect of the present invention, a topical pharmaceutical composition in the form of a vaginal gel ensures high efficacy due to local application.

The vaginal gel according to the present invention has the possibility of increasing the time of residence in situ, thus reducing the number of applications. Ideally, the formulation will be retained at the biological surface and the drug will be released close to the absorptive membrane, with a consequent enhancement of bioavailability.

The topical pharmaceutical composition of the present invention possesses lubricating properties and hence can be convenient during sexual intercourse. The degree of lubrication provided by the pharmaceutical composition of the present invention is an important determinant of its acceptability and use.

According to the present invention, a topical pharmaceutical composition in the form of a vaginal gel provides adequate lubrication so as to ensure patient compliance.

The topical pharmaceutical composition comprising tenofovir and lactic acid and optionally ciclopirox may further comprise suitable excipients that may be used for formulating the vaginal gel composition according to the present invention.

It will be understood that the composition of the invention will generally comprise one or more pharmaceutically acceptable excipients. Suitable excipients that may be used in the topical pharmaceutical composition include, but are not limited to gelling agents, chelating agents, preservatives, bioadhesives or polymers, viscosity modifiers or regulators, humectants/emollients, surfactants, pH adjusting agents, solvents/co-solvents and tonicity modifiers or osmolar agents.

Suitable gelling agents, that may be employed, in the topical pharmaceutical composition include, but are not limited to, xanthan gum, sodium alginate (Manugel DMB), Carbopol® ETD 2020, polycarbophil, polysaccharides, natural gums, acacia, tragacanth, starch, cellulose derivatives such as carboxy methyl cellulose, hydroxyl propyl methyl cellulose, hydroxypropyl methylcellulose (Methocel K4 M), methacrylate polymers, polyvinyl pyrrolidone, bentonite, alginic acid, carbomer, ethyl cellulose, gelatin, guar gum, hydroxyl ethyl cellulose, hydroxyl propyl cellulose, hydroxyethyl methylcellulose, glyceryl behenate, algae extracts, gums, polysaccharides, polyethylene oxide, poloxamer, pectins, hydrolysed proteins, polymers comprising pendant carboxylic acid groups, or esters thereof, polymers comprising pendant anhydrides of dicarboxylic acid groups and block co-polymers based on ethylene oxide and/or propylene oxide and the like or mixtures thereof.

Preferably, the one or more gelling agent may be present in an amount ranging from about 0.05% to about 10% by weight of the total composition.

Chelating agents that may be used in the topical pharmaceutical composition include, but are not limited to disodium edetate, condensed sodium phosphate, diethylenetriamine penta-acetic acid and the like or combinations thereof.

Preferably, the chelating agents may be present in an amount ranging from about 0.01% to about 1% by weight of the total composition.

Preservatives that may be used in the topical pharmaceutical composition include, but are not limited to hydroxybenzoates (parabens such as methyl paraben, propyl paraben), benzyl alcohol, benzoic acid, chlorphenesin, sorbic acid, phenoxyethanol and the like or combinations thereof.

Preferably, one or more preservatives may be present in an amount ranging from about 0.05% to about 2% by weight of the total composition.

Bioadhesives or polymers that may be used in the topical pharmaceutical composition, include, but are not limited to hydroxyethyl cellulose, gelatin, carbopol, polycarbophil, cross-linked polymethacrylic acid, hydroxypropyl methyl cellulose, hydroxypropyl cellulose, ethyl cellulose, polyethylene glycol, polysaccharide hyaluronic, polyvinylpyrrolidone, sodium alginate, sodium carboxymethylcellulose, methyl cellulose, starch and the like or combinations thereof.

Suitable humectants and/or emollients provide smoothness and lubricity which in turn facilitate the filling and dispensing of the topical pharmaceutical composition.

Emollients that may be used in the topical pharmaceutical composition, include, but are not limited to, polyhydric alcohols such as glycols, and polysaccharides, such as glycerin, ethylene glycol, propylene glycol, butylene glycol, diethylene glycol, dipropylene glycol, diglycerin, sorbitol, malvitol, trehalose, raffmose, xylitol, mannitol, polyethylene glycol, propylene glycol, polyglycerin, cholesterol, squaline, fatty acids, octyldodecanol, myristyl alcohol, urea, lanolin, lactic acid, esters such as isopropyl stearate, isopropyl myristate, isopropyl palmitate and isopropyl laurate and the like or combinations thereof.

Preferably, the emollients may be present in an amount ranging from about 2% to about 20% by weight of the total composition.

Viscosity modifiers or regulators improve the formation of a gel. Suitable viscosity modifiers or regulators that may be used in the topical pharmaceutical composition, include, but are not limited to, polyolefins, polyethylenes, polypropylenes, polyalphaolefins, ethylene-propylene copolymers, maleneated derivatives of the materials herein, polyisobutylenes, maleic anhydride and their diene derivatives, polymethacrylates, maleic anhydride-styrene copolymers and esters and their diene derivatives, hydrogenated copolymers of styrene-butadiene, ethylene-propylene copolymers, polyisobutenes, hydrogenated styrene-isoprene polymers, hydrogenated isoprene polymers, polymethacrylates, polyacrylates, polyalkyl styrenes, alkenyl aryl conjugated diene copolymers, polyolefins, esters of maleic anhydride-styrene copolymers, ethylene-propylene copolymers functionalized with the reaction product of maleic anhydride and an amine, polymethacrylate functionalized with an amine, styrene-maleic anhydride copolymers reacted with an amine, polymethacrylate polymers, esterified polymers, esterified polymers of a vinyl aromatic monomer and an unsaturated carboxylic acid or derivative thereof, olefin copolymers, ethylene-propylene copolymer, polyisobutylene and the like or combinations thereof.

Suitable tonicity modifiers or osmolar agents to match the osmolality (mosm) of the physiological fluids include, but are not limited to, glycerine, sodium chloride, potassium chloride, mannitol, sucrose, lactose, fructose, maltose, dextrose, dextrose anhydrous, propylene glycol, glycerol and the like or combinations thereof.

Suitable amphoteric, non-ionic, cationic or anionic surfactants may be included in the topical pharmaceutical composition of the present invention.

According to the present invention, surfactants may comprise, but are not limited to, one or more of coconut fatty acid diethanolamide, Polysorbates, Sodium dodecyl sulfate (sodium lauryl sulfate), Lauryl dimethyl amine oxide, Docusate sodium, Cetyl trimethyl ammonium bromide (CTAB) Polyethoxylated alcohols, Polyoxyethylene sorbitan, Octoxynol, N, N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt and the like or combinations thereof.

Preferably, one or more surfactants may be present in an amount ranging from about 0.05% to about 20% by weight of the total composition.

Suitable pH adjusting agents or buffering agents that may be used in the topical pharmaceutical composition, include, but are not limited to acidulants such as hydrochloric acid, acetic acid, citric acid, tartaric acid, propionic acid, sodium hydroxide, sodium phosphate, ammonia solution, triethanolamine, sodium borate, sodium carbonate, potassium hydroxide and the like or combinations thereof.

Preferably, one or more buffering agent may be present in an amount ranging from about 0.1% to about 2% by weight of the total composition.

Suitable solvents/co-solvents, solubilizer or vehicles, that may be employed, in the topical pharmaceutical composition include, but are not limited to, water, glycerine, coconut fatty acid diethanolamide, medium and/or long chain fatty acids or glycerides, monoglycerides, diglycerides, triglycerides, structured triglycerides, soyabean oil, peanut oil, corn oil, corn oil mono glycerides, corn oil di glycerides, corn oil triglycerides, polyethylene glycol, caprylocaproyl macroglycerides, caproyl 90, propylene glycol, polyoxyethylene sorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methylisobutyl ketone, methylethyl ketone or mixtures thereof.

Preferably, the one or more solvent may be present in an amount ranging from about 0.05% to about 20% by weight of the total composition.

The topical pharmaceutical composition, in this context, not only envisages compositions suitable for vaginal application but also compositions suitable for rectal and transdermal application under the ambit of the invention.

Formulations for rectal use may be presented as suppositories, retention enemas, ointments, creams, solutions, tablets, aerosols, jellies suspensions, gels or foams with suitable bases and transdermally as patches.

In the context of the present invention, it is to be understood that the term topical includes application to the body cavities as well as to the skin.

Thus, the topical pharmaceutical composition of the present invention, comprising tenofovir and lactic acid and optionally ciclopirox is applied to a body cavity such as the anus or the vagina. In a particularly preferred embodiment, the topical pharmaceutical composition is applied to the vagina.

The topical pharmaceutical composition of the present invention, comprising tenofovir and lactic acid and optionally ciclopirox may involve topical application to the vagina to facilitate contraception, prevent the transmission of retroviral infections and the treatment of bacterial vaginosis during vaginal intercourse. Thus, a composition according to the invention is provided for use in facilitating contraception and preventing retroviral infections and preventing or treating bacterial vaginosis.

Typically, the topical application is carried out prior to the beginning of vaginal intercourse, suitably 0 to 60 minutes, preferably 0 to 5 minutes prior to the beginning of vaginal intercourse.

In the present invention, the pH is suitably maintained between 3.0 and 4.5. Preferably, the pH of the topical pharmaceutical composition is between 3.8 and 4.2±0.2.

The topical pharmaceutical composition of the invention may further include sweeteners, fragrances and any such excipients which may improve the aesthetic appeal of the said composition.

In another aspect of the present invention the topical pharmaceutical composition may also be in the form of a nanoemulsion. Nanoemulsions are emulsions with mean droplet diameters ranging from 50 to 1000 nm and the droplet size between 100 and 500 nm. The particles can exist as water-in-oil and oil-in-water forms. Nanoemulsions can be obtained by any of the processes such as, but not limited, to high pressure homogenization, phase inversion temperature technique and microfluidization.

The nanoemulsions may comprise suitable excipients that may be used for formulating the nanoemulsions, such as, but not limited to oils, emulsifiers, antioxidants, tonicity modifiers, pH adjusting agents and preservatives.

In another aspect of the present invention the topical pharmaceutical composition may also be in the form of a nanosuspension. Nanosuspensions are very finely colloidal, biphasic dispersed solid drug particles in an aqueous vehicle, size below 1 μm.

In another aspect of the present invention, the topical pharmaceutical composition may also be in the form of solid lipid nanoparticles.

In another aspect of the present invention, the topical pharmaceutical composition may also comprise micelles. A micelle is an aggregate of surfactant molecules dispersed in a liquid colloid. When surfactants are present above the CMC (Critical micelle concentration), they can act as emulsifiers that will allow a compound that is normally insoluble (in the solvent being used) to dissolve.

In another aspect of the present invention the topical pharmaceutical composition may also be in the form in which the active moieties are released in response to some event such as vaginal or anal intercourse. For example, the composition may contain the composition in vesicles or liposomes, which are disrupted by the mechanical action of intercourse. Liposomes are microscopic vesicles in which a variety of drugs can be incorporated to form a non-toxic and biodegradable formulation because of the similarity of the primary components of liposomes with natural membranes. It allows high cellular penetration, efficient targeting of macrophage-rich tissues and a marked improvement in drug pharmacokinetics. The topical pharmaceutical composition according to the present invention provides improved delivery of active agents to the infected cells and also reduces the toxic effects associated with this composition which in turn exhibits improved efficacy and safety of the drug to facilitate contraception, prevent the transmission of retroviral infections and the treatment of bacterial vaginosis.

According to one embodiment of the present invention the topical pharmaceutical composition may comprise actives in a nanosize range.

Nanosizing leads to increase in the exposure of surface area of particles leading to an increase in the rate of dissolution. The nanoparticles of the present invention can be obtained by any process such as, but not limited to milling, precipitation, homogenization, spray-freeze drying, supercritical fluid technology, PRINT (Particle replication in non-wetting templates), capillary aerosol generator, ultrasonication and spray drying.

According to one embodiment of the present invention, a topical pharmaceutical composition in the form of a vaginal gel can also be used with one or more additional active ingredients of the antiretroviral class. These antiretrovirals can belong to any of the below classes of protease inhibitors, nucleoside reverse transcriptase inhibitors, nucleotide reverse transcriptase inhibitors, non nucleotide reverse transcriptase inhibitors and integrase inhibitors.

Suitable protease inhibitors (PIs) that may be employed in the topical pharmaceutical composition of the present invention may comprise saquinavir; ritonavir; nelfinavir; amprenavir; lopinavir, indinavir; nelfinavir; atazanavir; lasinavir; palinavir; tirpranavir; fosamprenavir; darunavir; tipranavir; N-cycloalkylglycines, α-hydroxyarylbutanamides; α-hydroxy-γ-[[(carbocyclic- or heterocyclic-substituted)amino)carbonyl]alkanamide derivatives; γ-hydroxy-2-(fluoroalkylaminocarbonyl)-1-piperazinepentanamides; dihydropyrone derivatives and α- and β-amino acid hydroxyethylamino sulfonamides; and N-aminoacid substituted L-lysine derivatives.

Suitable nucleoside reverse transcriptase inhibitors (NRTIs) that may be employed in the pharmaceutical composition of the present invention may comprise Zidovudine; didanosine; stavudine; lamivudine; abacavir; adefovir; lobucavir; entecavir; apricitabine; emtricitabine; zalcitabine; dexelvucitabine; alovudine; amdoxovir; elvucitabine;; phosphazid; racivir; stampidine; β-L-FD4 (also called β-L-D4C and named β-L-2′,3′-dicleoxy-5-fluoro-cytidene); DAPD, the purine nucleoside, (−)-β-D-2,6-diamino-purine dioxolane; and lodenosine (FddA), 9-(2,3-dideoxy-2-fluoro-β-D-threo-pentofuranosyl)adenine.

Suitable nucleotide reverse transcriptase inhibitors (NtRTIs) that may be employed in the pharmaceutical composition of the present invention may comprise adefovir.

Suitable non-nucleotide reverse transcriptase inhibitors (NNRTIs) that may be employed in the pharmaceutical composition of the present invention may comprise nevirapine, rilpivirine, delaviridine, efavirenz, etravirine, furopyridine-thiopyrimide; capravirine; 5-(3,5-dichlorophenyl)-thio-4-isopropyl-1-(4-pyridyl)methyl-1H-imidazol-2-ylmethyl carbonate); emivirine (1-(ethoxy-methyl)-5-(1-methylethyl)-6-(phenylmethyl)-(2,4(1H,3H)-pyrimidinedione); (+)-calanolide A and B, coumarin derivatives; dapivirine; 4-{4-[4-((E)-2-cyano-vinyl)-2,6-dimethyl-phenylamino]-pyrimidin-2-ylamino]-benzonitrile; 12-ethyl-8-[2-(1-hydroxy-quinolin-4-yloxy)-ethyl]-5-methyl-11,12-dihydro-5H-1,5,10,12-tetraaza-dibenzo[a,e]cycloocten-6-one; 7-bromo-3-[2-(2,5-dimethoxy-phenyl)-ethyl]-3,4-dihydro-1H-pyrido[1,2-a][-1,3,5]triazine-2-thione and 1-(5-bromo-pyridin-2-yl)-3-(2-thiophen-2-yl-ethyl)-thiourea.

Suitable integrase inhibitors that may be employed in the pharmaceutical composition of the present invention may comprise raltegravir, elvitegravir.

The antiretroviral agents of the present invention may be used in the form of salts or esters derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

The present invention also provides a process for preparing a topical pharmaceutical composition in the form of a vaginal gel, which process comprises
a) preparing a drug phase comprising tenofovir and at least one antibacterial agent along with one or more excipients selected from the group consisting of vehicles, pH adjusting agents, chelating agents, and gelling agents;
b) preparing a solution comprising ciclopirox, one or more vehicles, preservatives;
c) dispersing ciclopirox solution of step b) into the drug phase of step a) to form the gel.

The present invention also provides a method of contraception by applying a topical pharmaceutical composition comprising tenofovir and at least one or more antibacterial agents and optionally ciclopirox.

The present invention also provides a method of preventing the transmission of retroviral infections and the treatment of bacterial vaginosis by applying the topical pharmaceutical composition comprising tenofovir and at least one or more antibacterial agents and optionally ciclopirox.

The present invention also provides a use of the topical pharmaceutical composition comprising tenofovir and at least one or more antibacterial agents and optionally ciclopirox as a contraceptive agent.

The present invention also provides a use of the topical pharmaceutical composition comprising tenofovir and at least one or more antibacterial agents and optionally ciclopirox for preventing the transmission of retroviral infections and the treatment of bacterial vaginosis.

The following examples are for the purpose of illustration of the invention only and are not intended in any way to limit the scope of the present invention.

### EXAMPLE 1

| Sr. No. | Ingredients | % w/w |
|---|---|---|
| 1. | Tenofovir Base (PMPA) | 1.00 |
| 2. | Lactic acid | 1.00 |
| 3. | Disodium EDTA | 0.05 |
| 4. | Propyl paraben | 0.02 |
| 5. | Methyl paraben | 0.18 |
| 6. | Hydroxy Ethylcellulose | 2.50 |
| 7. | Glycerin | 20.0 |
| 8. | Purified water | q.s. to 100% |
| 9. | 10% w/w HCl | To adjust the |
| 10. | 10% w/w NaOH | pH between 3.8 |
| | | and 4.5. |

Preparation of Organic Phase:
1. Glycerin was heated.
2. Methyl paraben and propyl paraben were added and dissolved in the heated glycerin
3. The solution obtained in step 2 was cooled.
4. Hydroxy ethylcellulose was added and dispersed in the solution obtained from step 3 to obtain a thick dispersion.

Preparation of Drug Phase:
1. Disodium edetate was dissolved in water.
2. Lactic acid was added and dissolved in the solution obtained in step 1.
3. Tenofovir was dispersed in the solution obtained in step 2.
4. Sodium hydroxide and hydrochloric acid were used to adjust the pH from 3.8-4.5 till a solution of tenofovir is obtained.

Preparation of Gel:
1. Drug phase was added to the organic phase under continuous stirring to obtain the gel.

### EXAMPLE 2

| Sr. No. | Ingredients | % w/w |
|---|---|---|
| 1. | Tenofovir Base (PMPA) | 1.00 |
| 2. | Lactic acid | 3.00 |
| 3. | Disodium EDTA | 0.05 |
| 4. | Propyl paraben | 0.02 |
| 5. | Methyl paraben | 0.18 |
| 6. | Polycarbophil | 5.0 |
| 7. | Glycerin | 20.0 |
| 8. | Purified water | q.s. to 100% |
| 9. | 10% w/w HCl | q.s. to dissolve |
| | | tenofovir |
| 10. | 10% w/w NaOH | q.s. to adjust the |
| | | pH between 3.8 |
| | | and 4.5. |

Preparation of Organic Phase:
1. Glycerin was heated.
2. Methyl paraben and propyl paraben were added and dissolved in the heated glycerin.
3. The solution obtained in step 2 was cooled.
4. Polycarbophil was added and dispersed in the solution obtained from step 3 to obtain a thick dispersion.

Preparation of Drug Phase:
1. Disodium edetate was dissolved in water.
2. Lactic acid was added and dissolved in the solution obtained in step 1.
3. Tenofovir was dispersed in the solution obtained in step 2.
4. Sodium hydroxide and hydrochloric acid were used to adjust the pH from 3.8-4.5 till a solution of tenofovir is obtained.

Preparation of Gel:
1. Drug phase was added to the organic phase under continuous stirring to obtain the gel.

### EXAMPLE 3

| Sr. No | Ingredients | % w/w |
|---|---|---|
| 1 | Tenofovir | 1.00 |
| 2 | Lactic acid | 2.00 |
| 3 | Cyclopirox olamine | 1.00 |
| 4 | Glycerin | 10.00 |
| 5 | Propylene Glycol | 10.00 |
| 6 | Methyl paraben | 0.18 |
| 7 | Propyl paraben | 0.02 |
| 8 | Coconut fatty acid | 4.00 |
| | diethanolamide | |
| 9 | Polysorbate 60 | 5.00 |
| 10 | Xanthan gum | 3.00 |
| 11 | Disodium edetate | 0.05 |
| 12 | Citric acid monohydrate | 1.00 |
| 13 | 10% w/w Hydrochloric acid | q.s till tenofovir |
| | solution | dissolves |
| 14 | 10% w/w sodium hydroxide | q.s to (pH 3.8-4.0) |
| | solution | |
| 15 | Purified water | q.s to 100 % |

Preparation of Tenofovir Drug Phase
1) Di sodium edetate was dissolved in purified water.
2) Citric acid, lactic acid and tenofovir was added to the solution obtained in step (1).
3) Hydrochloric acid was added to the solution obtained in step (2) to dissolve tenofovir.
4) The pH of the solution obtained in step (3) was adjusted with sodium hydroxide solution.
5) Xanthan gum was added to the solution obtained in step (4) to form a lump free gel.

Preparation of Cyclopirox Olamine Solution
1) Glycerine and propylene glycol were added to purified water and heated.
2) Methyl paraben, propyl paraben, coconut fatty acid diethanolamide and polysorbate 60 were added to the solution obtained in step (1).

Preparation of Gel:
1) Drug phase was added to the organic phase under continuous stirring to obtain the gel and the required volume was made up with purified water and the pH was determined.

It will be readily apparent to one skilled in the art that varying substitutions and modifications may be made to the invention disclosed herein without departing from the scope of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

1. A topical pharmaceutical composition comprising tenofovir and at least one antibacterial agent and, optionally, ciclopirox.

2. The topical pharmaceutical composition according to claim 1 wherein the composition comprises ciclopirox.

3. The topical pharmaceutical composition according claim 1 wherein one or more of tenofovir, the at least one antibacterial agent and ciclopirox are in the form of a pharmaceutically acceptable derivative, which comprise pharmaceutically acceptable salts, solvates, complexes, hydrates, anhydrates, isomers, esters, tautomers, enantiomers, polymorphs, or prodrugs.

4. The topical pharmaceutical composition according to claim 1 wherein tenofovir is in the form of tenofovir disoproxyl fumarate or tenofovir alafenamide fumarate.

5. The topical pharmaceutical composition according to claim 1 wherein tenofovir is present in an amount ranging from about 1% to about 5% by weight of the total composition.

6. The topical pharmaceutical composition according to claim 1 wherein the antibacterial agent is selected from lactic acid, citric acid, fumaric acid, tartaric acid, malic acid, and acetic acid.

7. The topical pharmaceutical composition according to claim 1 wherein the antibacterial agent comprises lactic acid.

8. The topical pharmaceutical composition according to claim 1 wherein the antibacterial agent comprises lactic acid in the form of racemic lactic acid, D(−) lactic acid, or L(+) lactic acid.

9. The topical pharmaceutical composition according to claim 1 wherein lactic acid is present in an amount ranging from about 1% to about 10% by weight of the total composition.

10. The topical pharmaceutical composition according to claim 1 wherein ciclopirox is in the form of ciclopirox olamine.

11. The topical pharmaceutical composition according to claim 1 wherein ciclopirox olamine is present in an amount ranging from about 0.05% to about 5% by weight of the total composition.

12. The topical pharmaceutical composition according to claim 1 comprising tenofovir disoproxil fumarate and lactic acid and optionally ciclopirox olamine.

13. The topical pharmaceutical composition according to claim 1 comprising tenofovir alafenamide fumarate and lactic acid and optionally ciclopirox olamine.

14. The topical pharmaceutical composition according to claim 12, comprising ciclopirox olamine.

15. The topical pharmaceutical composition according to claim 1, comprising one or more pharmaceutically acceptable excipients selected from one or more of chelating agents, preservatives, bioadhesives, viscosity modifiers, humectants/emollients, surfactants and pH adjusting agents, and tonicity modifiers.

16. The topical pharmaceutical composition according to claim 1 wherein the composition is in a dosage form suitable for vaginal or rectal application.

17. The topical pharmaceutical composition according to claim 1 wherein composition is provided in the form of a gel, tablet, capsule, pessary, tampon, implant, cream, paste, jelly, foam, film, ring, spray, suppository, enema, ointment, solution or suspension.

18. The topical pharmaceutical composition according to claim 1 wherein the composition is in the form of a nanoemulsion or nanosuspension or as a solid lipid nanoparticulate or micelles.

19. The topical pharmaceutical composition according to claim 1 in the form of a vaginal or rectal gel.

20. The topical pharmaceutical composition according to claim 1 wherein the composition has a pH of from 3.0 to 4.5.

21. The topical pharmaceutical composition according to claim 1, further comprising at least one antiretroviral agent selected from protease inhibitors, nucleoside reverse transcriptase inhibitors, nucleotide reverse transcriptase inhibitors, non-nucleotide reverse transcriptase inhibitors and integrase inhibitors.

22. A process for preparing a topical pharmaceutical composition according to claim 1 in the form of a vaginal gel, which process comprises
a) preparing a drug phase comprising tenofovir and at least one antibacterial agent along with one or more pharmaceutically acceptable excipients.
b) preparing a ciclopirox solution along with one or more pharmaceutically acceptable excipients.
c) dispersing ciclopirox solution of step b) into the drug phase of step a) to form the gel.

23. A method of providing or facilitating contraception which method comprises the application of a topical pharmaceutical composition according to claim 1 to a patient in need thereof.

24. A method of treating retroviral infections or bacterial infections, particularly bacterial vaginosis, which method comprises the application of a topical pharmaceutical composition according to claim 1 to a patient in need thereof.

25. The topical pharmaceutical composition according to claim 1 for use as a medicament.

26. The topical pharmaceutical composition according to claim 1 for use as a contraceptive agent.

27. The topical pharmaceutical composition according to claim 1 for use in treating retroviral infections, or bacterial infections, particularly bacterial vaginosis.

28. (canceled)
